(19) United States
(12) Patent Application Publication
(10) Pub. No.: US 2021/0366578 A1
(43) Pub. Date: Nov. 25, 2021

(54) EMBRYONIC DNA REGISTRY

(71) Applicant: India Treniek Price, San Diego, CA (US)

(72) Inventor: India Treniek Price, San Diego, CA (US)

(21) Appl. No.: 16/882,471

(22) Filed: May 23, 2020

(51) Int. Cl. G16B 50/30; G06F 16/2457; G06F 16/2455; G16H 10/40; G16H 10/60

(52) U.S. Cl. CPC G16B 50/30; G06F 16/2457; G16H 10/60; G16H 10/40; G06F 16/24565

(57) ABSTRACT

A system and method for providing a registry database of DNA test results for an embryonic population is disclosed. The Embryonic DNA Registry is created and managed by a remote server communicating with laboratories and users using a web browser. DNA profiles from available embryos are generated by a laboratory and stored into the database that contains the registry. Users submit a sample to a laboratory to generate a DNA profile of the prospective parent(s) that is submitted to the registry server for searching. Users submit search queries that cause the server to compare the prospective parent DNA profiles with the DNA profiles in the registry. Results of matching DNA profiles that meet the search query are returned to the user. The remote server processing elements include a DNA profile matcher, a search report generator, a user account processor, a network interface, a test result analyzer, and a database engine.

### TECHNICAL FIELD

This application relates in general to a system and method for providing DNA testing, and more specifically, to a system and method for providing a registry database of DNA test results for an embryonic population according to the present invention.

### BACKGROUND

There are many people who are not capable of conceiving children by natural means and must resort to invitro-fertilization (IVF), and many more people exist who cannot afford to utilize IVF procedures. For those who can afford to utilize IVF procedures, an issue remains regarding what might be done with any unused embryos. Currently, these embryos are stored indefinitely or possibly destroyed. While it may be possible to adopt these unused embryos and have them implanted into other women and brought to full term, some people may fear providing the unused embryos to total strangers, or conversely, using the embryos of complete strangers. Many potential parents may wish to adopt children who may possess a biological connection to one or both of the adopting families.

Therefore, a need exists for a registry of DNA results obtained from unused embryos that may be searched using DNA from the potential adoptive parents to assist in identifying any available embryos with a possible connection to one or both of the adoptive families via the identification of embryos from related individuals such as siblings, aunts, cousins, second and third cousins, and so forth. Additionally, use of this registry may help adoptive parents find an available embryo with desirable ethnic or regional connections to one or more of their families. No such registry exists. The present invention attempts to address the limitations of currently available systems as disclosed herein.

### SUMMARY

In accordance with the present invention, the above and other problems are solved by providing a system and method for a registry database of DNA test results for an embryonic population according to the principles and example embodiments disclosed herein.

In one embodiment, the present invention is a system for providing a registry database of DNA test results for an embryonic population according to the present invention. The embryonic DNA registry is created and managed by a remote server communicating with laboratories and users via a web browser. DNA profiles from available embryos are generated by a laboratory and stored into the database registry. Potential adopters (users) submit a sample to a laboratory to generate a DNA profile of the prospective parents that is then submitted to the registry server for searching. Users submit search queries that cause the server to compare the prospective parents' DNA profiles with the DNA profiles in the registry. Results of matching DNA profiles that meet the search query are returned to the user. The remote server processing elements include a DNA profile matcher, a search report generator, a user account processor, a network interface, a test result analyzer, and a database engine.

In another embodiment, the present invention is a method for providing a registry database of DNA test results for an embryonic population according to the present invention. The method creates and stores an embryonic DNA profile record corresponding to an available embryo into the registry database, generates a set of search parameters defining a search query, submits the search query to the registry database, receives a search results report in response to the search query, selects an embryo identified in the search results report, and obtaining adoption information regarding the selected embryo.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only, and is not intended as a definition of the limits of the present invention.

### BRIEF DESCRIPTION OF THE DRAWINGS

Referring now to the drawings in which like reference numbers represent corresponding parts throughout:

FIG. 1a illustrates an example embodiment for a system that provides a registry database of DNA test results for an embryonic population according to the present invention.

FIG. 1b illustrates an example DNA Test Report with evidence of a possible family relationship according to the present invention.

FIG. 2a is a block diagram illustrating an exemplary hardware architecture of a computing device.

FIG. 2b is a block diagram illustrating an exemplary logical architecture for a client device.

FIG. 2c is a block diagram showing an exemplary architectural arrangement of clients, servers, and external services.

FIG. 2d is another block diagram illustrating an exemplary hardware architecture of a computing device.

FIG. 3 illustrates another example embodiment of a system for providing a registry database of DNA test results for an embryonic population according to the present invention.

FIG. 4 illustrates a computing system of software components providing a registry database of DNA test results for an embryonic population according to the present invention.

FIG. 5 illustrates a flowchart corresponding to a method performed by software components providing a registry database of DNA test results for an embryonic population according to the present invention.

### DETAILED DESCRIPTION

This application relates in general a system and method for providing DNA testing, and more specifically, to a system and method for providing a registry database of DNA test results for an embryonic population according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. Thus, for example, reference to “a needle” includes reference to one or more of such needles and “etching” includes one or more of such steps. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “50-250 micrometers” should be interpreted to include not only the explicitly recited values of about 50 micrometers and 250 micrometers, but also include individual values and sub-ranges within the indicated range. Thus, included in this numerical range are individual values such as 60, 70, and 80 micrometers, and sub-ranges such as from 50-100 micrometers, from 100-200, and from 100-250 micrometers, etc.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specifications and claims are approximations that may vary depending upon the desired properties sought to be obtained by the present disclosure. At the very least, and not as an attempt to limit the application of the doctrine of equivalents to the scope of the claims, each numerical parameter should be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in the testing measurements.

As used herein, the term “about” means that dimensions, sizes, formulations, parameters, shapes, and other quantities and characteristics are not and need not be exact, but may be approximated and/or larger or smaller, as desired, reflecting tolerances, conversion factors, rounding off, measurement error and the like, and other factors known to those of skill. Further, unless otherwise stated, the term “about” shall expressly include “exactly,” consistent with the discussion above regarding ranges and numerical data.

As used herein, the term “adenine” refers to one of the four nucleobases found in DNA. Adenine (A) always pairs with thymine (T).

As used herein, the term “allele” refers to an alternate sequence for a particular position in the genome.

As used herein, the term “ancestry” refers to a line of descent, therefore a person, plant, animal or object that is related to one existing at a later point in time.

As used herein, the term “chromosome” refers to any of the rod-like structures found in all living cells that contains the chemical patterns which control what an animal or plant is like.

As used herein, the term “cytosine” refers to one of the four nucleobases found in DNA. Cytosine (C) always pairs with guanine (G).

As used herein, the term “DNA” refers to deoxyribonucleic acid: the double-stranded molecule at the center of the cells of living things which controls the structure and purpose of each cell and carries genetic information during reproduction.

As used herein, the term “embryo” refers to multi-cellular unborn human offspring in the process of development after fertilization. The term embryo herein refers to one or more embryos.

As used herein, the term “gene” refers to the basic unit of heredity: a sequence of DNA nucleotides on a chromosome.

As used herein, the term “gene frequency” refers to the relative occurrence of a particular allele in a population.

As used herein, the term “gene mapping” refers to the determination of the relative positions of genes on a DNA molecule (chromosome or plasmid) and of the distance, in linkage units or physical units, between them.

As used herein, the term “guanine” refers to one of the four nucleobases found in DNA. Guanine (G) always pairs with cytosine (C).

As used herein, the term “locus (pl. loci)” refers to the specific physical location of a gene on a chromosome.

As used herein, the term “marker” refers to the gene of known location on a chromosome and phenotype that is used as a point of reference in the mapping of other loci.

As used herein, the term “RNA” refers to ribonucleic acid: the single-stranded molecule present in all living cells that acts as a messenger carrying instructions from DNA.

As used herein, the term “sequencing” refers to the determination of the order of nucleotides (base sequences) in a DNA or RNA molecule or the order of amino acids in a protein.

As used herein, the term “thymine” refers to the pyrimidine base; one of the four molecules containing nitrogen present in the nucleic acids DNA and RNA; thymine (T) always pairs with adenine (A).

As used herein, the term “X-chromosome” refers to the sex chromosome which exists as a pair in the cells of females and with a Y-chromosome in the cells of male animals.

As used herein, the term “Y-chromosome” refers to the sex chromosome that exists only in male cells.

The terms “couple,” “prospective parent(s),” “potential adopter(s)” and “user” refer to an entity, e.g. a human, using the Embryonic DNA Registry including any software or smart device application(s) associated with the invention. Any of these terms may be used interchangeably to refer to these individual(s). The term user herein refers to one or more users.

The term “connection” refers to connecting any component as defined below by any means, including but not limited to, a wired connection(s) using any type of wire or cable for example, including but not limited to, coaxial cable(s), fiberoptic cable(s), and ethernet cable(s) or wireless connection(s) using any type of frequency/frequencies or radio wave(s). Some examples are included below in this application.

The term “invention” or “present invention” refers to the invention being applied for via the patent application titled “Embryonic DNA Registry.” Invention may be used interchangeably with registry.

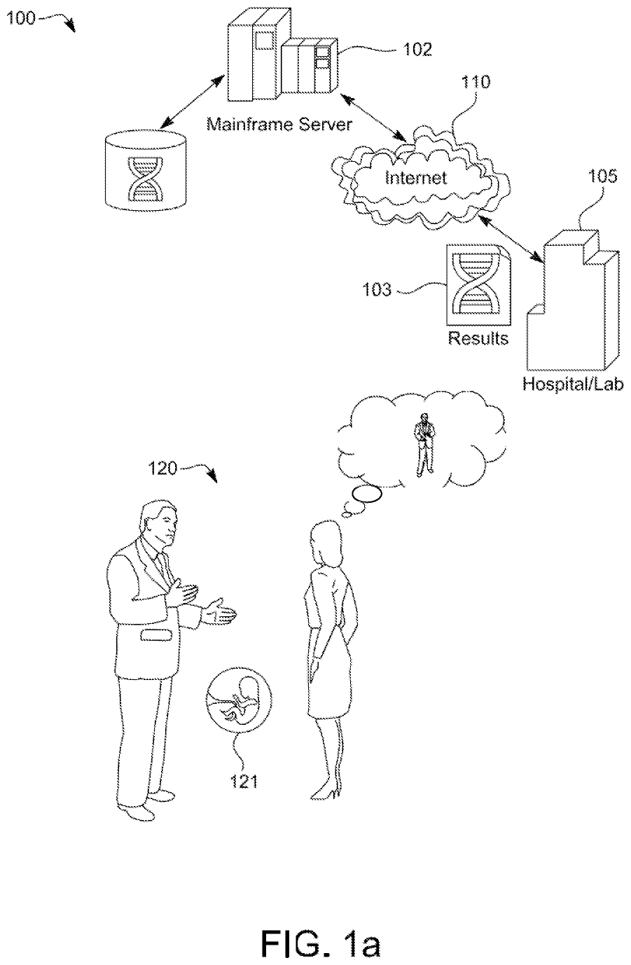

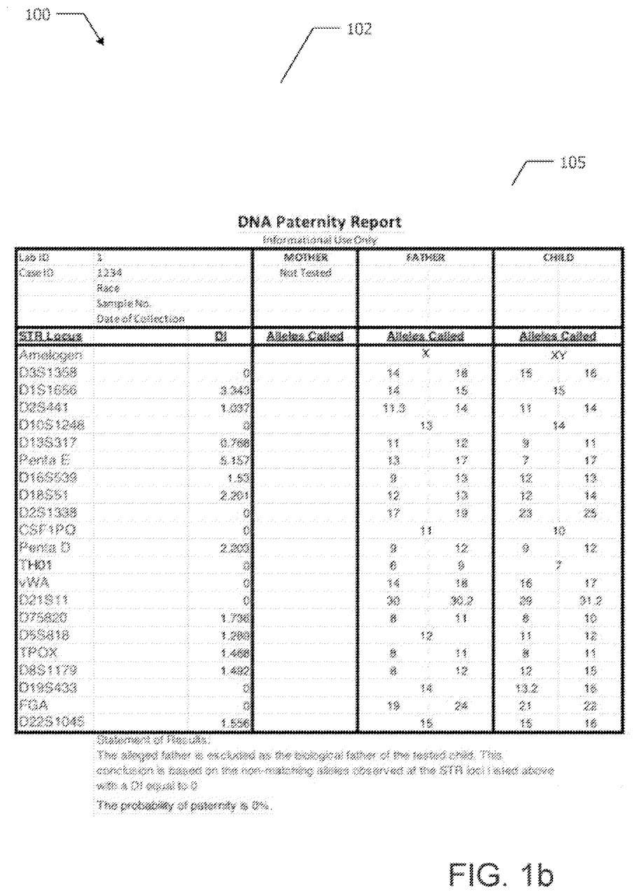

In general, the present disclosure relates a system and method for providing DNA testing. To better understand the present invention, FIG. 1a illustrates an example embodiment for a system 100 providing a registry database of DNA test results for a population of embryos available for adoption according to the present invention. A couple 120 seeking to adopt an embryo 121 may wish that their child be related to someone in one or both of their families or that their child's biological parents were from a particular region of the world that may provide a common ethnic background. This couple 120 may have a DNA analysis performed on DNA samples taken from an ordinary swab of their inner cheek. This sample may be processed in a hospital or laboratory 105 in a typical manner as samples are currently processed by commercial entities such as 23andme.com™, ancestry.com™, National Geographic™, and similar services.

The analysis results 103 of a parent's DNA may be sent via the Internet 110 to a registry server 102 for matching against similar DNA profiles obtained from existing embryos available for adoption. When an embryo becomes available for adoption, an agency responsible for the health and safety of the embryo and that facilitates the adoption may harvest a few cells from the embryo to generate a DNA profile that is stored in the embryonic DNA registry 101. This DNA profile may have been prepared for the biological parents during an IVF process that they undertook that resulted in the creation of the available embryos and if so, the profile may be merely added to the registry 101.

In either case, the prospective couple 120 may have their DNA profile results 103 compared with all of the DNA profiles in the registry to find an available embryo that is related in some manner to the couple. When an embryo is identified, the couple 120 may be directed to a service that may request the selected embryo be made available, implant the selected embryo for a pregnancy, and proceed with any required legal adoption processes necessary to be viewed as the legal parents of the child. These steps may be governed by federal, state, and local laws and may require that the legal proceeding conclude before implantation of the embryo may occur.

The registry 101 may provide the parent(s) 120 with a search process in which they may specify any particular parameters to be used in matching their DNA profiles with embryos' DNA profiles from the registry 101. If no matching embryo is found to be available using a set of search parameters, the couple 120 may change or broaden the search in some manner such as searching for a connection more than a set number of prior generations that may be identified from the DNA profiles. This iterative process may continue until either an embryo is selected, or until the parent(s) 120 terminate the search activity. The identity of the biological parents may be protected and not provided to searchers before or after a selection occurs. The privacy of donors may be protected as they may have specified at the time of the donation. The adopting parent(s) 120 may not be as concerned about the identity of the biological parents for an embryo they choose to adopt when they have been provided a mechanism to ensure that the embryo possesses ties of their definition to the adopter(s).

The DNA Test Report, shown in FIG. 1b, shows the results of laboratory DNA tests that provide evidence regarding an alleged family relationship. This is a brief explanation of the meaning of the numbers (that is to say, the statistical certainty of the result) and other items that appear in a DNA test report:

The laboratory analysis tests the DNA isolated from cheek swabs to locate certain regions of chromosomes that are known to vary in length between individuals. Twenty-four of these sites are tested; each site is called a “locus”, (“loci”—plural). Analysis of these 24 sites in a large population has revealed many different-sized versions associated with each site. Versions of a DNA sequence or a gene are called “alleles.” These are the genetic markers used in grandparents DNA testing.

Because each individual has two of each type of chromosome, one inherited from each parent, everyone has two alleles at each locus. These two alleles are sometimes identical (homozygous), but usually they are not the same size (heterozygous). During parentage DNA testing, the laboratory identifies the length of the two alleles found at each locus.

The DNA test report 105 of FIG. 1b shows numbers (in the first column) that indicate each of the 21 loci involved in the DNA testing process.

The columns marked “allele” on the DNA test report contain numbers indicating the two alleles found at each locus (or one number if they are the same size).

If, for example, a child has two alleles that are designated 12.1 and 18, and if the mother has alleles 12.1 and 16, then the child inherited the 12.1 allele from the mother. The child has to have inherited the 18 allele from the father. The 18 allele is the “obligate paternal allele.” Generally, an alleged father must have this allele if he is the biological father of the child.

Twenty (20) different loci are used as genetic markers in the DNA tests, as well as one (acerogenin) to confirm the gender of the person providing the DNA sample. If the alleged father does not have the matching allele at every tested locus, then he usually cannot be the biological parent. If he does have it, then he could be the father. A “relationship index” (called the “Direct Index” in the report) for each locus is calculated based on information including the portion of the male population that has the obligate paternal allele at that locus. This index is reported for each DNA locus.

A combined relationship, or Direct Index, for all of the tested alleles is then calculated and appears below the chart. This number is used to calculate the “probability of relationship,” which is the percentage likelihood that a man with the alleles of the alleged father is the biological parent of the child, as compared to an untested, unrelated man of the same race. If the DNA of the alleged father is consistent (to a degree of mathematical certainty) with that of the child, then the report will conclude that the alleged father cannot be excluded as the biological father of the child. If the DNA is not consistent however, it will conclude that the alleged father can be excluded as the biological father of the child.

Each locus used in DNA testing is composed of a variable number of repeating short sequences of the DNA building blocks or “nucleobases.” These are referred to in short-hand as “A,” “C,” “T,” and “G.” An example of a natural DNA sequence having such simple short repeats would be ACGACGACGACG, i.e. (ACG)4. However, each allele can have a different number of total repeats, for example (ACG)3 or (ACG)5 instead of (ACG)4, giving rise to a different fragment length when the DNA is purified and amplified in the test tube. In this example, (ACG)3, (ACG)4 and (ACG)5 would encode variant alleles with building block lengths of 3×3, 4×3, and 5×3, that is to say 9, 12, and 15 building block lengths. Since DNA analysis machines detect changes in length of just one unit, these differences in length are easily distinguished.

The above example of comparing a child's DNA profile with a father's DNA profile is representative of the comparison performed using embryonic DNA profiles in the registry 101 and the adopter's DNA profile(s). While the above example presents a determination that a parent-child relationship exists between the two DNA profiles, similar comparisons may be performed that look for relationships that span multiple generations before a common ancestor is found. The reports are generated by comparing a DNA sample result with each of the sample records stored within the registry. Sample records having a matching value greater than a predefined threshold may be returned in results provided to a client in response to a search.

The invention may use any type of network such as a single network, multiple networks of a same type, or multiple networks of different types which may include one or more of a direct connection between devices, including but not limited to a local area network (LAN), a wide area network (WAN) (for example, the Internet), a metropolitan area network (MAN), a wireless network (for example, a general packet radio service (GPRS) network), a long term evolution (LTE) network, a telephone network (for example, a Public Switched Telephone Network or a cellular network), a subset of the Internet, an ad hoc network, a fiber optic network (for example, a fiber optic service (often known as FiOS) network), or any combination of the above networks.

Smart devices mentioned herein the present application may also use one or more sensors to receive or send signals, such as wireless signals for example, Bluetooth™, wireless fidelity, infrared, Wi-Fi, or LTE. Any smart device mentioned in this application may be connected to any other component or smart device via wired communications (e.g., conductive wire, coaxial cable, fiber optic cable, ethernet cable, twisted pair cable, transmission line, waveguide, etc.), or a combination of wired and wireless communications. The invention's method and/or system may use a single server device or a collection of multiple server devices and/or computer systems.

The systems and methods described above, may be implemented in many different forms of applications, software, firmware, and hardware. The actual software or smart device application codes or specialized control software, hardware or smart device application(s) used to implement the invention's systems and methods is not limiting of the implementation. Thus, the operation and behavior of the systems and methods were described without reference to the specific software or firmware code. Software, smart device application(s), firmware, and control hardware can be designed to implement the systems and methods based on the description herein.

While all of the above functions are described to be provided to users via a mobile application on a smartphone, one of ordinary skill will recognize that any computing device including tablets, laptops, and general purpose computing devices may be used as well. In at least one embodiment, all of the services described herein are provided using web pages being accessed from the web server 201 using a web browser such as Safari™, Firefox™, Chrome™ DuckDuckGo™, and the like. All of the screen examples described herein show user interface elements that provide the functionality of the present invention. The arrangement, organization, presentation, and use of particular user input/output (I/O) elements including hyperlinks, buttons, text fields, scrolling lists, and similar I/O elements are shown herein for example embodiments only to more easily convey the features of the present invention. The scope of the present invention should not be interpreted as being limited by any of these elements unless expressly recited within the attached claims.

For the purposes of the example embodiment of FIG. 1, various functions are shown to be performed on different programmable computing devices that communicate with each other over the Internet 105. These computing devices may include smartphones 101a, laptop computers 101b, tablets (not shown), and similar devices so long as the disclosed functionality of the mobile application described herein is supported by the particular computing device. One of ordinary skill will recognize that this functionality is grouped as shown in the embodiment for clarity of description. Two or more of the processing functions may be combined onto a single processing machine. Additionally, it may be possible to move a subset of processing from one of the processing systems shown here and retain the functionality of the present invention. The attached claims recite any required combination of functionality onto a single machine, if required, and all example embodiments are for descriptive purposes.

For all of the above devices that are in communication with each other, some or all of them need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects, and in order to more fully illustrate one or more aspects. Similarly, although process steps, method steps, algorithms or the like may be described in a sequential order, such processes, methods, and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method or algorithm is carried out or executed. Some steps may be omitted in some aspect or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other aspects need not include the device itself.

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing modules, segments or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC) or on a network interface card.

Software/hardware hybrid implementations of at least some of the aspects disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific aspects, at least some of the features or functionalities of the various aspects disclosed herein may be implemented on one or more general-purpose computers associated with one or more networks, such as for example, an end-user computer system, a client computer, a network server or other server system, a mobile computing device (e.g., tablet computing device, mobile phone, smartphone, laptop or other appropriate computing device), a consumer electronic device, a music player or any other suitable electronic device, router, switch or other suitable device, or any combination thereof. In at least some aspects, at least some of the features or functionalities of the various aspects disclosed herein may be implemented in one or more virtualized computing environments (e.g., network computing clouds, virtual machines hosted on one or more physical computing machines or other appropriate virtual environments).

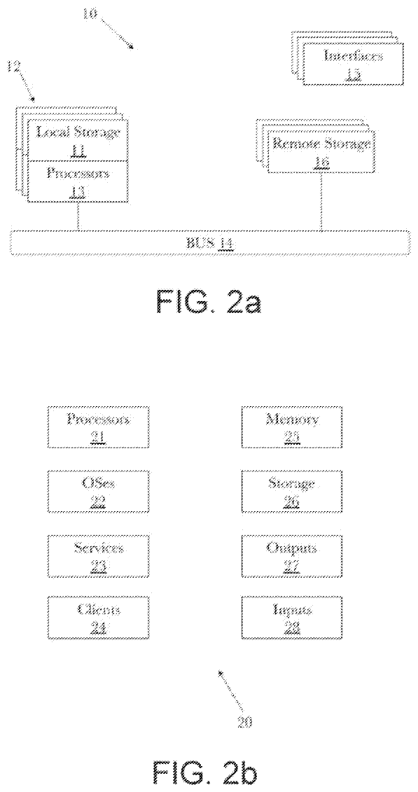

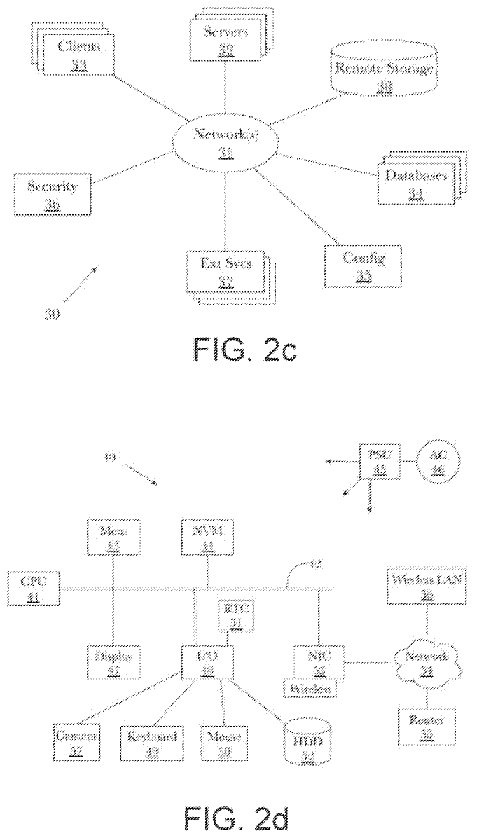

Referring now to FIG. 2a, there is a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network, a metropolitan area network, a local area network, a wireless network, the Internet or any other network, using known protocols for such communication, whether wireless or wired.

In one aspect, computing device 10 includes one or more central processing units (CPU) 12, one or more interfaces 15, and one or more buses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing a CPU 12, local memory 11 and/or remote memory 16, and interface(s) 15. In at least one aspect, a CPU 12 may be caused to perform one or more of the different types of functions and/or operations under the control of software modules or components, which for example, may include an operating system and any appropriate applications software, drivers, and the like.

A CPU 12 may include one or more processors 13 such as for example, a processor from one of the Intel, ARM, Qualcomm, and AMD families of microprocessors. In some aspect, processors 13 may include specially designed hardware such as application-specific integrated circuits (ASICs), electrically erasable programmable read-only memories (EEPROMs), field-programmable gate arrays (FPGAs), and so forth, for controlling operations of a computing device 10. In a particular aspect, a local memory 11 (such as non-volatile random access memory (RAM) and/or read-only memory (ROM), including for example, one or more levels of cached memory) may also form part of a CPU 12. However, there are many different ways in which memory may be coupled to a system 10. Memory 11 may be used for a variety of purposes such as, for example, caching and/or storing data, programming instructions, and the like. It should be further appreciated that a CPU 12 may be one of a variety of system-on-a-chip-(SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may, for example, support other peripherals used with a computing device 10. Among the interfaces that may be provided are ethernet interfaces, frame relay interfaces, cable interfaces, DSL interfaces, token ring interfaces, graphics interfaces, and the like. In addition, various types of interfaces may be provided such as, for example, universal serial bus (USB), serial, ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast ethernet interfaces, gigabit ethernet interfaces, serial ATA (SATA) or external SATA (ESATA) interfaces, high-definition multimedia interfaces (HDMI), digital visual interfaces (DVI), analog or digital audio interfaces, asynchronous transfer mode (ATM) interfaces, high-speed serial interfaces (HSSI), point of sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 2a illustrates one specific architecture for a computing device 10 for implementing one or more of the aspects described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, a single processor 13 handles communications as well as routing computations, while in other aspects a separate dedicated communications processor may be provided. In various aspects, different types of features or functionalities may be implemented in a system according to the aspect that includes a client device (such as a tablet device or smartphone running client software) and a server system (such as a server system described in more detail below).

Regardless of network device configuration, the system of an aspect may employ one or more memories or memory modules (for example, remote memory block 16 and local memory 11) configured to store data, program instructions for the general-purpose network operations or other information relating to the functionality of the aspects described herein (or any combinations of the above). Program instructions may control execution of or comprise an operating system and/or one or more applications, for example. Memory 16 or memories 11, 16 may also be configured to store data structures, configuration data, encryption data, historical system operations information or any other specific or generic non-program information described herein.

Because such information and program instructions may be employed to implement one or more systems or methods described herein, at least some network device aspects may include non-transitory machine-readable storage media, which, for example, may be configured or designed to store program instructions, state information, and the like for performing various operations described herein. Examples of such non-transitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device) or they may be removable such as swappable flash memory modules (such as “thumb drives” or other removable media designed for rapidly exchanging physical storage devices), “hot-swappable” hard disk drives or solid state drives, removable optical storage disks, or other such removable media, and that such integral and removable storage media may be utilized interchangeably. Examples of program instructions include both object code, such as may be produced by a compiler, machine code, such as may be produced by an assembler or a linker, byte code, such as may be generated by for example by a JAVA™ compiler and may be executed using a JAVA™ virtual machine or equivalent, or files containing higher level code that may be executed by the computer using an interpreter (for example, scripts written in Python™, Perl™, Ruby™, Groovy™, or any other scripting language).

In some aspects, systems may be implemented on a standalone computing system. Referring now to FIG. 2b, there is a block diagram depicting a typical exemplary architecture of one or more aspects or components thereof on a standalone computing system. A computing device 20 includes processors 21 that may run software that carry out one or more functions or applications of aspects, such as for example a client application 24. Processors 21 may carry out computing instructions under control of an operating system 22 such as, for example, a version of MICROSOFT WINDOWS™ operating system, APPLE macOS™ or iOS™ operating systems, some variety of the LINUX™ operating system, ANDROID™ operating system or the like. In many cases, one or more shared services 23 may be operable in a system 20, and may be useful for providing common services to client applications 24. Services 23 may, for example, be WINDOWS™ services, user-space common services in a LINUX™ environment or any other type of common service architecture used with an operating system 21. Input devices 28 may be of any type suitable for receiving user input including, for example, a keyboard, touchscreen, microphone (for example, for voice input), mouse, touchpad, trackball or any combination thereof. Output devices 27 may be of any type suitable for providing output to one or more users, whether remote or local to system 20, and may include, for example, one or more screens for visual output, speakers, printers or any combination thereof. Memory 25 may be RAM having any structure and architecture known in the art for use by processors 21, for example to run software. Storage devices 26 may be any magnetic, optical, mechanical, memristor or electrical storage device for storage of data in digital form (such as those described above, referring to FIG. 2a). Examples of storage devices 26 include flash memory, magnetic hard drive, CD-ROM, and the like.

In some aspects, systems may be implemented on a distributed computing network, such as one having any number of clients and/or servers. Referring now to FIG. 2c, there is a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in Fig. b. In addition, any number of servers 32 may be provided for handling requests received from one or more clients 33. Clients 33 and servers 32 may communicate with one another via one or more electronic networks 31, which may be in various aspects any Internet, wide area network, mobile telephony network (such as CDMA or GSM cellular networks), wireless network (such as WiFi, WiMAX, LTE, and so forth) or local area network (or indeed any network topology known in the art; the aspect does not prefer any one network topology over another). Networks 31 may be implemented using any known network protocols, including, for example, wired and/or wireless protocols.

In addition, in some aspects, servers 32 may call external services 37 when needed to obtain additional information, or to refer to additional data concerning a particular call. Communications with external services 37 may take place, for example, via one or more networks 31. In various aspects, external services 37 may comprise web-enabled services or functionality related to or installed on the hardware device itself. For example, in one aspect where client applications 24 are implemented on a smartphone or other electronic device, client applications 24 may obtain information stored on a server system 32 in the Cloud or on an external service 37 deployed on one or more of a particular enterprise's or user's premises. In addition to local storage on servers 32, remote storage 38 may be accessible through the network(s) 31.

In some aspects, clients 33 or servers 32 (or both) may make use of one or more specialized services or appliances that may be deployed locally or remotely across one or more networks 31. For example, one or more databases 34 in either local or remote storage 38 may be used or referred to by one or more aspects. It should be understood by one having ordinary skill in the art that databases in storage 34 may be arranged in a wide variety of architectures and use a wide variety of data access and manipulation means. For example, in various aspects one or more databases in storage 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some aspects, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system or a logical database within an overall database management system. Unless a specific meaning is specified for a given use of the term “database,” it should be construed to mean any of these senses of the word, all of which are understood as a plain meaning of the term “database” by those having ordinary skill in the art.

Similarly, some aspects may make use of one or more security systems 36 and configuration systems 35. Security and configuration management are common information technology (IT) and web functions, and some amount of each are generally associated with any IT or web system. It should be understood by one having ordinary skill in the art that any configuration or security subsystems known in the art now or in the future may be used in conjunction with aspects without limitation, unless a specific security 36 or configuration system 35 or approach is required by the description of any specific aspect.

FIG. 2d shows an exemplary overview of a computer system 40 as may be used in any of the various locations throughout the system. It is exemplary of any computer that may execute code to process data. Various modifications and changes may be made to a computer system 40 without departing from the broader scope of the system and method disclosed herein.

A CPU 41 is connected to a bus 42, to which bus is also connected to memory 43, nonvolatile memory 44, display 47, I/O unit 48, and network interface card (NIC) 53. An I/O unit 48 typically may be connected to peripherals such as a keyboard 49, pointing device 50, hard disk 52, real-time clock 51, camera 57, and other peripheral devices. A NIC 53 connects to a network 54, which may be the Internet or a local network, which local network may or may not have connections to the Internet. The system may be connected to other computing devices through the network via a router 55, wireless local area network 56 or any other network connection. Also shown as part of a system 40 is a power supply unit 45 connected, in this example, to a main alternating current (AC) supply 46. Not shown are batteries that could be present and many other devices and modifications that are well known, but are not applicable to, the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles or other integrated hardware devices).

In various aspects, functionality for implementing systems or methods of various aspects may be distributed among any number of client and/or server components. For example, various software modules may be implemented for performing various functions in connection with the system of any particular aspect, and such modules may be implemented to run on server and/or client components.

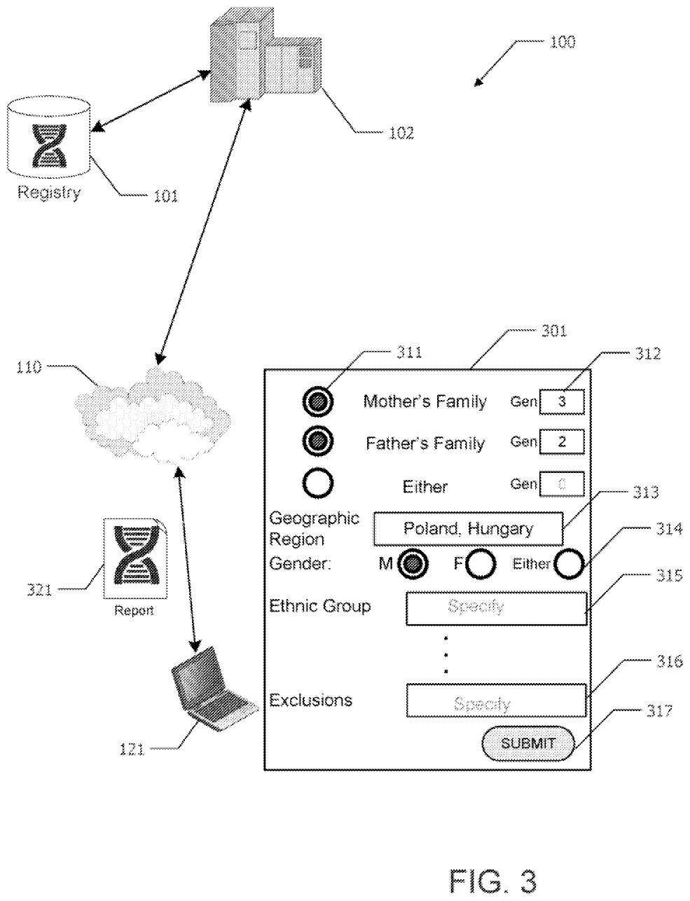

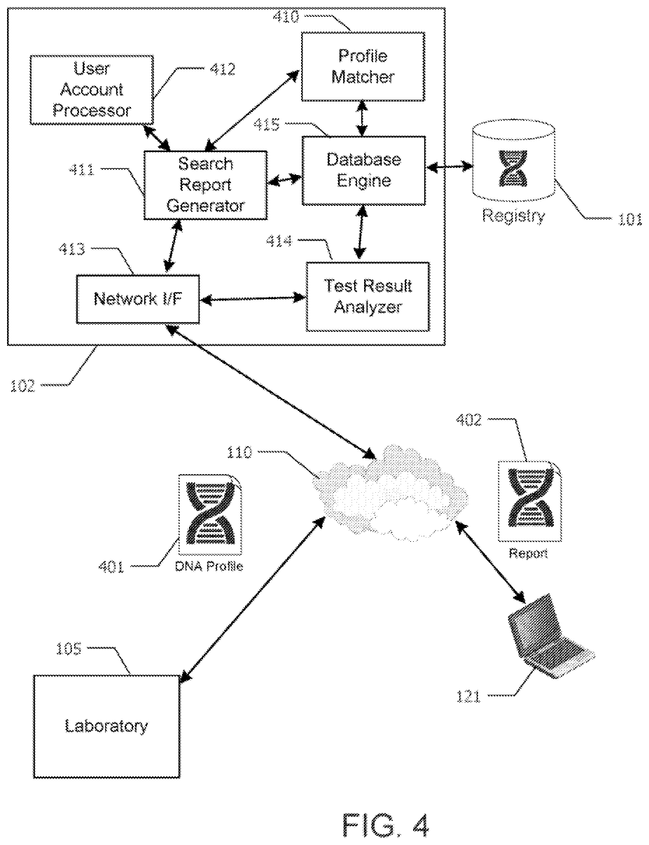

FIG. 3 illustrates another example embodiment of a system for providing a registry database of DNA test results of a population according to the present invention according to the present invention. In this figure, the user/couple 120 are performing a search of the registry 101. The couple 120 may upload one or both of their own DNA profiles to the registry 101 and its server 102. The couple 120 also specifies a set of search parameters 301, and specifies which parameters are to be used in the search. The search parameters comprise searching the mother's family 311, searching the father's family, searching a specified geographic area 313, searching for a child's gender 314, searching an ethnic group 315, and specifying any exclusions requested 316. Other parameters may also be used when comparing a couple's DNA profile against all of the embryo DNA profiles in the registry 101.

Once the search parameters are defined, the user may submit the search to the registry 101 using an input button or similar user interface device. These parameters are sent to the server 102 for processing. The request will identify the prospective parent's DNA profile from a user ID used to access the server 102. The server 102 processes the request by comparing the prospective parent's DNA profile against all of the DNA embryo profiles in the registry 101. The comparison to be made in the search as similar to the comparison of FIG. 1b made between a child and a potential father. In this search, the comparison is attempting to match the embryo's DNA profile against a likely relative of the prospective parent(s). The comparison is looking for matching allele at a number of loci to identify matching family members.

Once the server 102 completes is search of the registry 101, the user receives a report 321. This report identifies any available embryo matching the submitted search parameters any may include any estimate of a match that may be determined from the data within the compared DNA profiles. The report 321 may also indicate if no DNA embryonic DNA profiles were found to have matched the particular search query. The prospective parents may modify the search parameters and submit the updated query to search for additional matching DNA profiles. The search process may continue until the adopter(s) 120 decide to select a particular embryo for possible adoption.

A selected embryo will generate contact information regarding the appropriate servicing agency having access to the selected embryo. The adopter's information may be directly sent to the servicing agency as confirmation. Presumably, the selected DNA profile may be marked in the registry as having a pending selection until any financial and legal requirements are completed to permit the selected embryo to be provided to the adopter(s) 120. If the adopter(s) 120 complete these financial and legal requirements, the selected embryo is made available to the adopter(s) 120, the doctor of record or to a facility to perform the IVF implantation. At this point in time, the selected DNA profile for the selected embryo may be removed from the registry 101 as is should no longer be considered available for adoption.

The above embodiment contemplates the DNA profiles to be contained in the registry 101 correspond to embryos that are made available for adoption. This system is addressing the problem associated with leftover and unwanted embryos created by one couple as part of their IVF treatment such that these embryos may be adopted and brought to full term by parent(s) willing to adopt, raise, and love them. One of ordinary skill will recognize that other sources of DNA for searching may also be used by the present invention and the invention should not be so limited. The present invention is defined solely by the claims attached herein.

FIG. 4 illustrates a computing system of software components providing a registry database of DNA test results for an embryonic population according to the present invention. In this embodiment, users 120 may use a personal computer 121 to access the registry server 102 when searching the registry 101. This access may simply be a secure connection to a web page using an ordinary browser over the Internet 110. Results of searches may be provided as a responsive web page that may be viewed by the users using a web browser as well. The report of matching DNA profiles may also be returned to the users 120 using other communications methods such as email, SMS messages, telephone calls, USPS letters, and possible combinations of one or more of these methods. For example, the report 402 may take a long time to generate depending upon the number of DNA profiles to be compared with the adopter(s) 120 DNA profile(s). As such, the actual results may be made available for viewing using a web page; however, the results will be accessed after an email or similar message has been sent to the couple 120 indicating that the results are ready for viewing. As part of this process, multiple authentication factors may be utilized to ensure that the results are only made available to the potential adopter(s) 120 and no one else.

As noted above, embryonic DNA reports corresponding to available embryos are generated by a laboratory 105 and transmitted to the registry server 102 for inclusion in the registry 101. Similarly, adopter(s) 120 submit samples to the laboratory 105 to have DNA profiles generated and sent to the server 102. Once all of the data is available, the adopter(s) log into the server 102 and submits one or more search queries that compare the adoptive DNA profile(s) with the registry 101.

The registry server 102 performs the search operation processing to generate a search report 402. The registry server 102 uses a set of processing elements 410-415 to perform the search. The registry server 102 processing elements include a DNA profile matcher 410, a search report generator 411, a user account processor 412, a network interface 413, a test result analyzer 414, and a database engine 415. The DNA profile matcher 410 compares a DNA profile from one or both of the prospective parents 120 in a manner described above with reference to FIG. 1b and FIG. 3. The DNA profile matcher 410 retrieves each of the DNA profiles for available embryos in the registry 101 via the database engine 415 and generates a set of data to be used with the search parameters submitted by the adopter(s) 102. The results of these comparisons are sent to the search report generator 411.

The search report generator 411 receives results of the comparison of the prospective parent(s)′ DNA profile with the embryo profiles from the registry 101. The search report generator 411 applies the search parameters to these results and saves results meeting the match criteria set forth in the search query. This process continues until all DNA profiles from the registry have been checked. All of the saved results are formatted into a final report that is made available to the prospective parent(s) 120. These results will include a unique reference ID for each available embryo, all relevant search comparison results showing why the comparison matches the search query, and all relevant information useful for the prospective parent(s) to select a particular embryo for adoption as well as proceed to the next step to complete the adoption.

The user account processor 412 generates a user account for the potential adopter(s) 120 at the start of the search process. The user account processor 412 maintains copies of the DNA profiles for the adopter(s) 120 once received. The user account processor 412 is responsible for authenticating the adopter(s) 120 when he/she logs into the server 102 to perform a search, receive search results, and to select a particular embryo for adoption. The log in process may use a user ID and password and may also use other multiple-factor authorization mechanisms such as sending a unique temporary code to the user who is logging in to the server 102 via a phone number or email address that must be submitted by the user before the authentication process completes. This temporary code is typically valid for a short period of time and is randomly generated at the time of login. The phone number and email addresses to be used in the multi-factor process are specified by the user when the user account is created.

The network interface 413 connects the server 102 to the Internet 110 to send and receive communication from users, laboratories, hospitals, doctor's offices, and the like. The network interface 413 performs all necessary data formatting, data packet creation, data encryption for security, and data transmission and reception when the server 102 communicates with other processing systems disclosed herein. The network interface 413 is also responsible to ensure reception of any communications to other computing systems and to log any errors or attempts to hack into the registry.

The test result analyzer 414 pre-processes all DNA data profiles received for insertion into the registry 101 and use as prospective parent DNA profiles as disclosed herein. The test result analyzer 414 parses and formats DNA profile data received from any number of different testing laboratories into proper form for inclusion into the registry database 101. Any data translation, including language translations, required to make DNA profile data useful for searching the registry database 101 are performed once when the data is first received and thus permitting the other processing elements to utilize a common format and language regardless of the source of the DNA profile data.

The database engine 415 processes all database operations for the registry 101. These operations include insertion of embryonic DNA profiles into the database 101, deletion of embryonic DNA profiles from the database 101, searching and retrieving embryonic DNA profiles from the database 101, and indexing the database 101 to maintain efficient searching when needed.

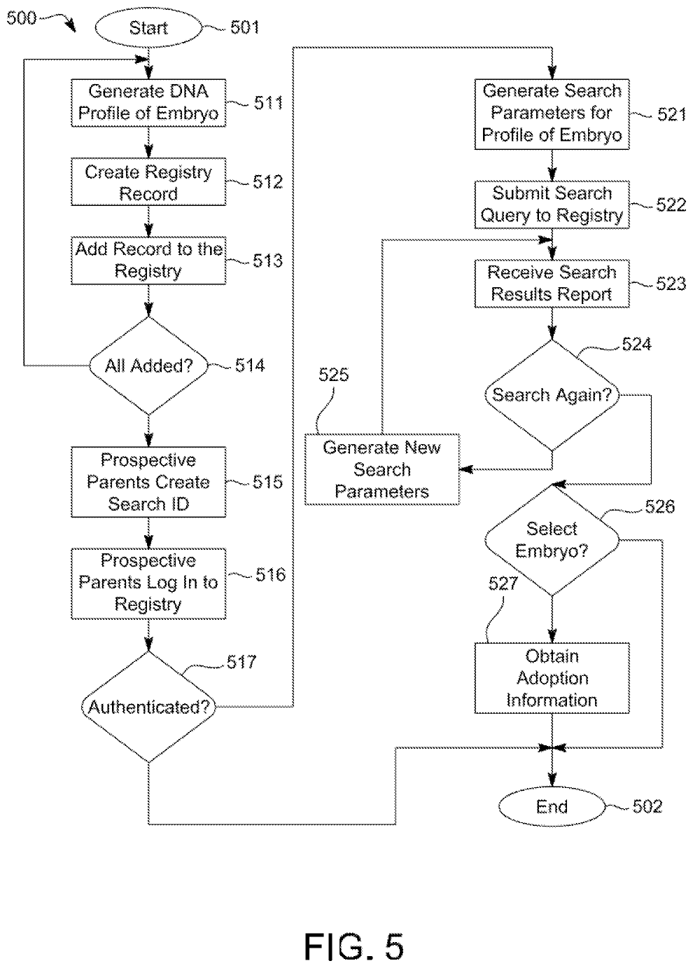

FIG. 5 illustrates a flowchart corresponding to a method performed by software components providing a registry database of DNA test results for an embryonic population according to the present invention. The process 500 begins 501 and step 511 generates a DNA profile for an available embryo. This step requires a laboratory to harvest DNA from cells extracted from the embryo and then to process the DNA material to generate a DNA profile containing sequenced DNA data. All of the generated data in used to create a registry record in step 512. This registry record typically includes a unique reference ID, the sequenced DNA data, a status indication of the embryo being available or being selected for adoption, servicing agency identification data, and any other useful information used when searching the database and comparing the registry record to an adopter(s)′ DNA profile(s).

The completed record is inserted into the registry in step 513 before test step 514 determines whether additional embryonic DNA profiles are to be stored into the registry. If test step 514 determines there are additional profiles to be processed, the process returns to step 511 to begin processing the next profile; otherwise the process continues to step 515 where an adopter creates a search user account.

Once an account exists, the potential adopter(s) may log into the registry server 102 in step 516 as described herein. Test step 517 determines whether the user has been successfully authenticated, and if not, the process ends 502; otherwise the user, in step 521 creates a search query. The search query contains all search parameters defined by the user. The search query is submitted to the registry server 102 in step 522, permitting the server 102 to perform the search and generate a report that is returned to the user in step 523.

Test step 524 determines whether the user wishes to modify the search query parameters to perform additional searching, and if so, step 525 modifies the search query parameters based upon input from the user. The updated query is submitted to the registry in step 523 and the process continues until the user is satisfied with the result. When the search is completed, test step 524 determines whether the user wishes to select a particular embryo for additional steps to adopt it. If the test step 524 receives an indication that an embryo is being selected, the user obtains all adoption information in step 525 before the process ends; otherwise the process immediately ends.

The embodiments described herein are implemented as logical operations performed by a computer. The logical operations of these various embodiments of the present invention are implemented (1) as a sequence of computer-implemented steps or program modules running on a computing system and/or (2) as interconnected machine modules or hardware logic within the computing system. The implementation is a matter of choice dependent on the performance requirements of the computing system implementing the invention. Accordingly, the logical operations making up the embodiments of the invention described herein can be variously referred to as operations, steps or modules.

Even though particular combinations of features are recited in the present application, these combinations are not intended to limit the disclosure of the invention. In fact, many of these features may be combined in ways not specifically recited in this application. In other words, any of the features mentioned in this application may be included to this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise. Any singular term used in this present patent application is applicable to its plural form even if the singular form of any term is used.

In the present application, all or any part of the invention's software or application(s) or smart device application(s) may be installed on any of the user's or operator's smart device(s), any server(s) or computer system(s) or web application(s) required to allow communication, control or transfer of content(s) or data between any combination of the components

What is claimed:

1. A system for providing DNA testing, and more specifically, to a system for providing a registry database of DNA test results for an embryonic population, the system comprising:
a search report generator for selecting comparisons of one or more embryonic DNA profiles from the registry database and an adopter DNA profile for inclusion into a DNA report using a search query;
a user account processor for authenticating users accessing the registry database to submit the search query; and
a DNA profile matcher for processing a comparison of one or more embryonic DNA profiles from the registry database and an adopter DNA profile;
wherein the database registry contains embryonic DNA profiles corresponding to embryos available for adoption.

2. The system according to claim 1, wherein the system further comprises:
a test results analyzer for pre-processing DNA profiles received from a laboratory for use within the system; and
a database engine coupled to the registry database for processing all database operations for the registry including insertion of embryonic DNA profiles into the registry database, deletion of embryonic DNA profiles from the registry database, searching and retrieving embryonic DNA profiles from the registry database, and indexing the registry database to maintain efficient searching when needed.

3. The system according to claim 2, wherein the user account processor further maintains adopters' DNA profile for use by the DNA profile matcher.

4. The system according to claim 1, wherein the adopters submits the search query to the registry database using a remote computing system over the Internet.

5. The system according to claim 1, wherein the search query includes one or more search values comprising use of a first adopter's DNA profile, use of a second adopter's DNA profile, use of specified geographic regions, use of one or more specified ethnic groups, and one or more specified exclusions.

6. The system according to claim 5, wherein use of the first adopter's DNA provide matches one or more of the adopter's DNA profiles looking back a specified number of generations.

7. The system according to claim 5, wherein use of the second adopter's DNA provide matches one or more of the adopter's DNA profiles looking back a specified number of generations.

8. A method for providing DNA testing, and more specifically, to a method for providing a registry database of DNA test results for an embryonic population, the method comprising:
creating and storing an embryonic DNA profile record corresponding to an available embryo into the registry database;
generating a set of search parameters defining a search query;
submitting the search query to the registry database;
receiving a search results report in response to the search query;
selecting an embryo identified in the search results report; and
obtaining adoption information regarding the selected embryo.

9. The method according to claim 8, wherein the creating and storing an embryonic DNA profile record corresponding to an available embryo into the registry database comprising:
generating an embryonic DNA profile corresponding to the available embryo for adoption;
creating the embryonic DNA profile record using the embryonic DNA profile corresponding to the available embryo for adoption and adoption information associated with the available embryo; and
adding the embryonic DNA profile record to the registry database.

10. The method according to claim 9, wherein method further comprising:
after receiving the search results report, modifying the set of search parameters defining the search query; and
resubmitting the search query to the registry database causing the generation of a new search results report.
